(19) United States
(12) Patent Application Publication
(10) Pub. No.: US 2019/0142448 A1
(43) Pub. Date: May 16, 2019

(54) GRASPER WITH INCREASED GRASPING SURFACE AREA

(71) Applicant: MOR Research Applications Ltd., Tel Aviv (IL)

(72) Inventor: Igor Igov, Ramla (IL)

(21) Appl. No.: 16/098,474

(22) PCT Filed: May 4, 2017
(86) PCT No.: PCT/IB2017/052584

§ 371 Date: Nov. 2, 2018

Related U.S. Application Data
(60) Provisional application No. 62/332,899, filed on May 6, 2016.
(60) Provisional application No. 62/404,239, filed on Oct. 5, 2016.

(51) Int. Cl. A61B 17/29

(52) U.S. Cl. CPC A61B 17/29; A61B 2017/2943; A61B 2017/2939

(57) ABSTRACT

The subject matter discloses a grasper, comprising a grasping element at a distal end of the grasper; wherein the grasping element is configurable to at least a narrow state having a narrow cross-section, and at least an expanded state having an expanded cross-section; wherein the grasping element configured in the narrow state is insertable through an incision or narrow orifice into a body; wherein the grasping element configured in the expanded state has an increased grasping surface; and wherein the grasping element is configurable from the narrow state to the expanded state after insertion into the body.

### CROSS-REFERENCE TO RELATED APPLICATIONS

This application claims priority from U.S. Provisional Patent Application No. 62/332,899 filed on 6 May 2016, titled “WIDENED HEAD”, and of U.S. Provisional Patent Application No. 62/404,239, titled “GRASPER WITH INCREASED GRASPING SURFACE AREA” filed on 5 Oct. 2016, both of which are expressly incorporated herein by reference in their entirety.

### FIELD OF THE INVENTION

The present disclosure generally relates to a grasper, according to exemplary embodiments.

### BACKGROUND

Minimally invasive surgeries (“MIS”)—sometimes referred to as keyhole surgeries, such as laparoscopic surgery or thoracoscopic surgery, are performed in a manner that attempts to minimize the amount of intrusion and invasion required for completion of the MIS. MIS typically involve one or more relatively small (e.g. about 2 to 12 mm) incisions in a patient's body, e.g. the abdominal area, to provide entry points for various surgical instruments including cutting, grasping and positioning instruments as well as viewing or imaging devices to enable the physician to perform the surgery. To minimize the invasiveness of the surgery, medical devices and surgical tools are designed to be as small as possible, thus minimizing or at least reducing the size of the required incisions for inserting the medical devices. To achieve a minimal incision size that is sufficient for successful MIS, the medical devices, e.g. cameras, scissors, forceps, graspers, probes, dissectors, hooks, retractors, and the like are designed to be as small as possible.

Graspers specifically are used to grasp tissue during the MIS and hold it in a manner that enables access to the area that is to be treated without having the tissue interfering or blocking access.

References considered to be relevant as background to the presently disclosed subject matter are listed below:

[1] US 2011/190653

[2] US 2012/150176

[3] U.S. Pat. No. 5,797,941

[4] US 2005/171560

[5] US 2014/0166720

[6] US 2009/270789

[7] US 2013/226200

[8] US 2008/119880

[9] U.S. Pat. No. 9,204,870

Acknowledgement of the above references herein is not to be inferred as meaning that these are in any way relevant to the patentability of the presently disclosed subject matter.

Moreover, the description above is presented as a general overview of related art in this field and should not be construed as an admission that any of the information it contains constitutes prior art against the present patent application

### SUMMARY

It is an object of the subject matter to disclose a grasper, comprising a grasping element at a distal end of the grasper; wherein the grasping element is configurable to at least a narrow state having a narrow cross-section, and at least an expanded state having an expanded cross-section; wherein the grasping element configured in the narrow state is insertable through an incision or narrow orifice into a body; wherein the grasping element configured in the expanded state has an increased grasping surface; and wherein the grasping element is configurable from the narrow state to the expanded state after insertion into the body, e.g., from outside the body by the user of the device.

In some cases, the grasping element is configurable from the expanded state back to the narrow state prior to extraction of the grasping element from the body.

In some cases, the grasping element comprises a first grasping jaw portion; a second grasping jaw portion; a connecting member coupling the first grasping jaw portion and the second grasping jaw portion; a control mechanism operably coupled to the distal end of the first grasping jaw portion wherein the first grasping jaw portion rotates around the connecting member when the control mechanism is activated; wherein the rotation causes a distal end of the first grasping jaw portion to pivot around an axis that is perpendicular to a longitudinal axis of the grasper, such that the distal end of the first grasping jaw portion becomes, after completing the rotation, adjacent to a proximal end of the second grasping jaw portion; a grasping surface comprising grasping ridges of the first grasping jaw portion adjacent to grasping ridges of the second grasping jaw portion.

In some cases, the grasper further comprising a handle member at a proximal end of the grasper for controlling the operation of the grasper a first elongated body member connected to the handle member; a second elongated body member coupling the handle member and with the grasping element; wherein the first elongated body member comprises a hollow body in which the second elongated body member is movable from the proximal end to the distal end within the hollow body; wherein actuating handles control the movement of the second elongated body member.

In some cases, the control mechanism is a push-pull lever.

In some cases, the grasping element comprises: a top grasping arm; a plurality of top grasping ridges coupled to the top grasping arm; a bottom grasping arm; a plurality of bottom grasping ridges coupled to the bottom grasping arm; a control mechanism connecting to the plurality of top grasping edges to extend the plurality of top grasping edges; wherein the control mechanism is connected to the plurality of bottom grasping edges to extend the plurality of bottom grasping edges.

In some cases, the plurality of top grasping edges and plurality of bottom grasping edges extend along a longitudinal axis.

In some cases, the plurality of top grasping edges and plurality of bottom grasping edges extend along a latitudinal axis.

In some cases, the grasper further comprising a protruding edge for puncturing a surface.

In some cases, the grasping element comprises: a top grasping element; wherein the top grasping element comprises: a first top grasping portion; a second top grasping portion; a top cavity enabling the first top grasping portion and the top second grasping portion to move when a top wedge is inserted into the top cavity; a bottom grasping element; and, wherein the bottom grasping element comprises: a first bottom grasping portion; a second bottom grasping portion; a bottom cavity enabling the first bottom grasping portion and the second bottom grasping portion to move when a bottom wedge is inserted into the bottom cavity.

In some cases, the grasper further comprising: an external elongated body member connected to the handle member; an internal elongated body member coupling the handle member and the grasping element; wherein the external elongated body member comprises a hollow body in which the internal elongated body member is movable from the proximal end to the distal end within the hollow body; wherein the actuating handles control the movement of the internal elongated body member.

It is another object of the subject matter to disclose a method performed using a grasping element comprising: configuring the grasping element to a narrow state having a narrow cross-section; inserting the grasping element into an incision in a body; configuring the grasping element to an expanded state for allowing a grasping operation, the expanded state having an expanded grasping surface area for grasping tissue without damaging said tissue; upon completion of the grasping operation, configuring the grasping element from the expanded state back to the narrow state; and removing the grasping element from the incision.

In some cases, the length of the connecting member determines the grasping surface area of the grasper.

In some cases, the grasping element is configured to open automatically when it is pushed out the distal end of the external elongated body member, and further wherein the grasping element closes when it is pulled back into the external elongated body member.

In some cases, the grasping element is controllably openable and closeable.

In some cases, the grasping element comprises elastically deformable material.

In some cases, the grasping element comprises selectively actuatable shape memory material.

In some cases, the grasping element comprises a top grasping element, a bottom grasping element, and a mechanical energy storage device, wherein the mechanical energy storage device is arranged and configured to impart an expanding force on the top and the bottom grasping elements such that the elements are pushed away from each other

This Summary introduces a selection of concepts in a simplified form that are further described below in the Brief Description of the Figures and the Detailed Description. This Summary is not intended to identify key features or essential features of the claimed subject matter, nor is it intended to be used to limit the scope of the claimed subject matter.

### BRIEF DESCRIPTION OF THE FIGURES

Some non-limiting exemplary embodiments or features of the disclosed subject matter are illustrated in the following drawings.

Identical, duplicate, equivalent or similar structures, elements, or parts that appear in one or more drawings are generally labeled with the same reference numeral, optionally with an additional letter or letters to distinguish between similar entities or variants of entities, and may not be repeatedly labeled and/or described.

Dimensions of components and features shown in the figures are chosen for convenience or clarity of presentation and are not necessarily shown to scale or true perspective. For convenience or clarity, some elements or structures are not shown or shown only partially and/or with different perspective or from different point of views. References to previously presented elements are implied without necessarily further citing the drawing or description in which they appear.

The number of elements shown in the Figures should by no means be construed as limiting and is for illustrative purposes only.

FIGS. 1A-1B schematically illustrate a grasper, according to exemplary embodiments of the disclosed subject matter;

FIGS. 2A-2T schematically illustrate a first exemplary embodiment of a grasping element of a grasper, according to the disclosed subject matter;

FIG. 3 shows a flow chart illustrating operation of a grasper, according to some exemplary embodiments of the subject matter;

FIGS. 4A-4E schematically illustrate a second exemplary embodiment of a grasping element of a grasper, according to the disclosed subject matter;

FIGS. 5A-5D schematically illustrate a third exemplary embodiment of a grasping element of a grasper, according to the disclosed subject matter; and,

FIGS. 6A-6F schematically illustrate a fourth exemplary embodiment of a grasping element of a grasper, according to the disclosed subject matter.

It is noted that the term “exemplary” is used herein to refer to examples of embodiments and/or implementations, and is not meant to necessarily convey a more-desirable use-case.

### DETAILED DESCRIPTION

In the context of some embodiments of the present disclosure, without limiting, a grasper is/implies a medical device configured to grab, clasp, grip and/or hold living tissue, for example during laparoscopic surgery.

In the context of some embodiments of the present disclosure, without limiting, the grasper may comprise a variety of grasping surfaces with which to grab tissue, the goal of said surface is to provide the optimal surface area and contact to grasp the tissue without fear of the grasper detaching. The grasping surface may be configured to form a surface shaped as ridges, teeth, undulations, protrusions, blades, or any combination thereof.

In the context of the present disclosure, without limiting, a minimally invasive surgery (“MIS”) is/implies any medical procedure conducted by applying minimal incisions to a human body. By limiting or reducing the size of incisions needed, the MIS allows reduction of wound healing time, associated pain and risk of infection. Examples of MIS include, but are not limited to, laparoscopic or arthroscopic surgery, endovascular aneurysm repair, angioplasty, endoscopy, etc.

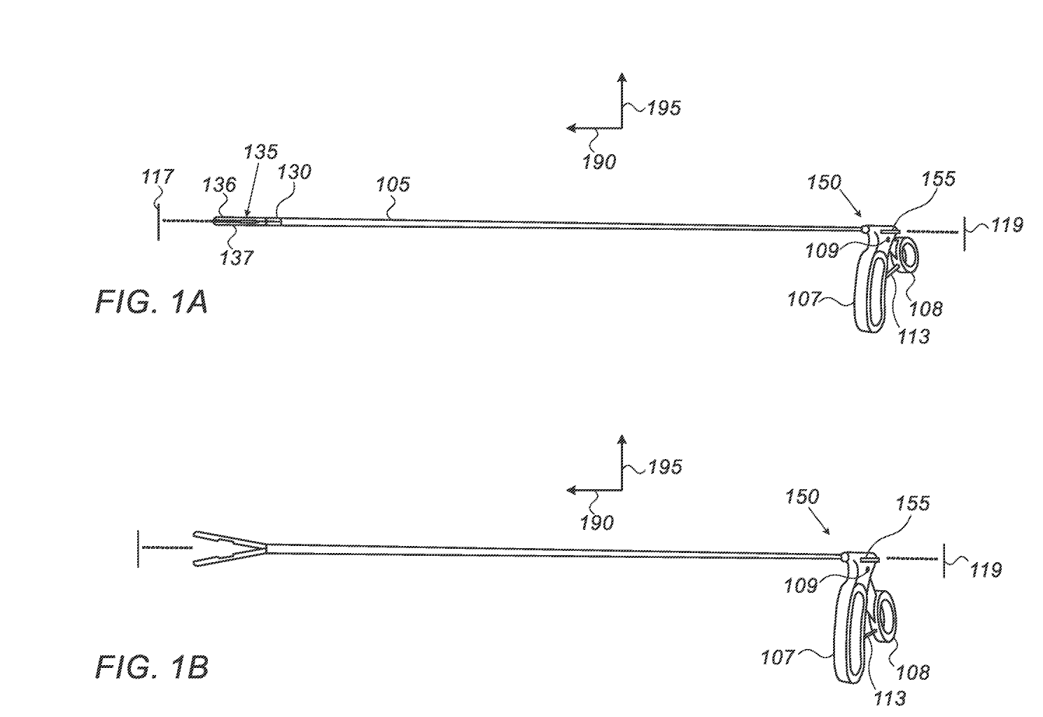

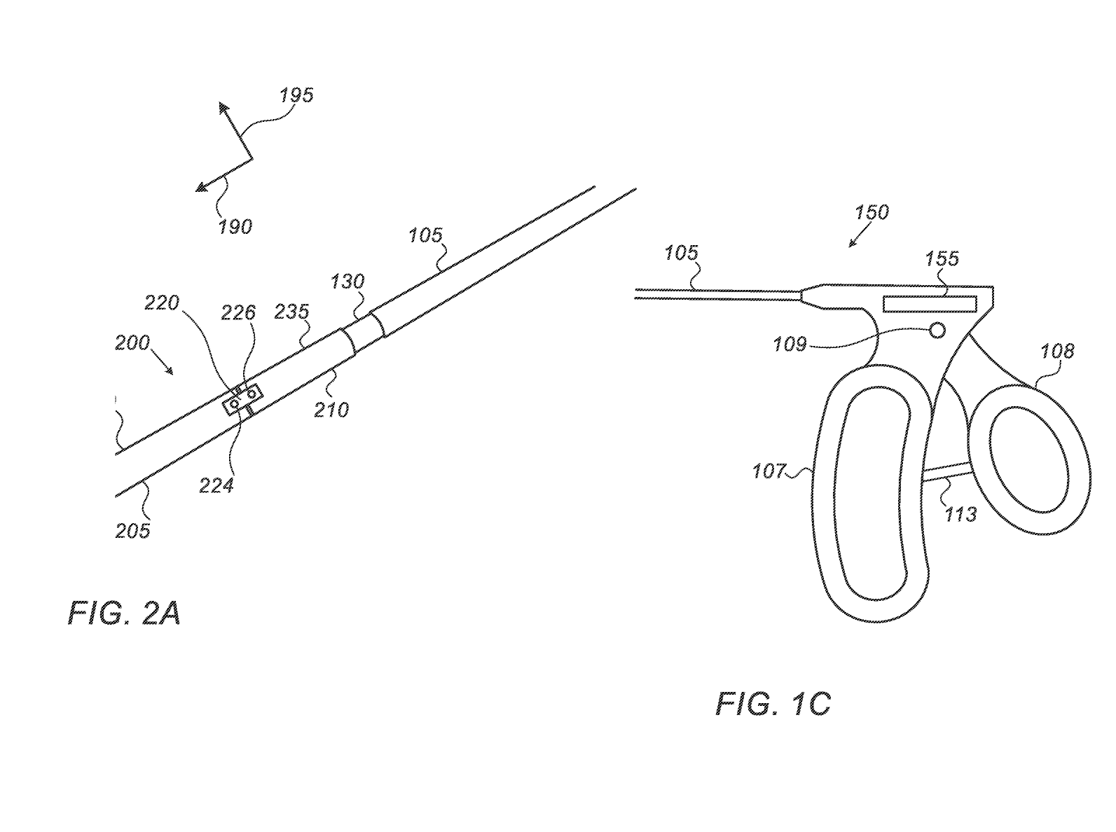

In the context of the present disclosure, without limiting, a proximal end is an end of the grasper situated nearest to a user of the grasper, e.g. during normal use of the grasper. For example, the proximal end of the grasper may include a handle member as further disclosed herein in FIGS. 1A-1B.

In the context of the present disclosure, without limiting, a distal end is an end of the grasper situated farthest from the user of the grasper, e.g. during normal use of the grasper. For example, the proximal end of the grasper may include a grasping jaw or arm of the grasper as further disclosed herein in FIGS. 1A-1B.

In the context of the present disclosure, without limiting, a longitudinal direction and a latitudinal direction is relative to the grasper such that the longitudinal direction is along a longitudinal axis of the grasper, and the latitudinal direction is perpendicular to the longitudinal axis, along a latitudinal axis of the grasper. For brevity and clarity and without limiting, in the present disclosure a grasper is for use during a MIS.

The terms cited above denote also inflections and conjugates thereof.

One exemplarily technical problem dealt by the disclosed subject matter is providing an increased contact surface area of a grasper, while the grasper maintains a small circumference or diameter or cross-section, in order to allow maintaining a minimal penetration footprint during insertion and/or extraction of the grasper from a body during a medical procedure. The penetration footprint, as referred to herein, may be or may be associated with a puncture width, incision width or length or area, diameter, and/or a cross-section or a cross section area (e.g. skin area), which is required and/or affected due to the penetration of the grasper into the body tissue. The terms cross-section and/or penetration footprint are used interchangeably herein. One advantage of maintaining a small circumference, diameter, cross section or penetration footprint of the grasper's portion that it is inserted into the body with a small puncture opening for insertion of the grasper and allows performing the medical procedure in the body. In the available graspers used in MIS procedures, due to the small diameter of the grasper (e.g. ranging between 2.2 mm-3 mm), the corresponding surface area of the grasper jaws intended for grabbing tissue is also small, which may for example increase the risk of unwanted puncturing, cutting and/or tearing of the tissue. Additionally, a small surface area may for example provide difficulty in maintaining a proper hold of the tissue during the MIS medical procedure.

One exemplarily technical solution according to the disclosed subject matter is providing several exemplary embodiments of graspers, which include transformable configurations of a grasping element between a narrow or insertion/extraction state, and an expanded or a grasping state. In the narrow state, the grasping element is configured to be small or minimal, for example, having a narrow cross-section in a range of 1 millimeter (mm)-5 mm, or 1.5 mm-3 mm, to enable insertion of the grasping element into small incisions. In the expanded state, the grasping element is configured to provide an expandable surface area of the grasping jaws or of the grasping element of the grasper while the grasper to hold tissue or some other desired object. The grasper provides for an expanded grasping surface comprising an expanded cross section once the grasping jaw has entered the body and is to be used, for example, grasping the tissue during a MIS. In some non-limiting exemplary embodiments, in the expanded stated at least one dimension of the grasping jaws is extendable or expandable, e.g. cross-section of the grasping element may be expanded from an initial penetration size, for example, within a range of 1.5 mm-3 mm, to an increased grasping cross section, for example, within a range of 3 mm-6 mm to provide the expanded grasping surface.

A general non-limiting presentation of practicing the present disclosure is given below, outlining exemplary practice of embodiments of the present disclosure and providing a constructive basis for variant and/or alternative and/or divergent embodiments, some of which are subsequently described.

FIGS. 1A-1C schematically illustrate a grasper, according to exemplary embodiments of the disclosed subject matter. FIG. 1A schematically illustrates the grasper 100, according to some exemplary embodiments of the subject matter. The grasper 100 comprises an external elongated body member 105, an internal elongated body member 130, and, optionally, a handle member 150 that includes actuating handles 107 and 108 at a proximal end 119, and a grasping element 135 at a distal end 117.

The grasping element 135 comprises grasping jaws or arms, for example, grasping arms 136 and 137. The grasping arms 136 and 137 open and close along a latitudinal axis 195 according to actuation by a user of the actuating handles 107 and 108. The grasper 100 comprises a securing mechanism 109, which secures the actuating handles 107 and 108 and the grasping element 135 in a desired position. For example, securing the grasping element 135 in a closed state when the grasping element 135 is grasping tissue, prevents the grasping element 135 from accidentally releasing the tissue during a MIS.

In some non-limiting cases, the securing mechanism 109 may lock the grasping arms 136 and 137, for example via a security pin locking the actuating handles 107, 108 in a desired position, when the security pin is inserted into a hole or a depression (not shown) in the actuating handles 107, 108. In other non-limiting embodiments, the securing mechanism 109 may comprise a ratchet 113, which is inserted in between the actuating handles 107, 108 and prevents the movement of the actuating handles 107, 108.

The external elongated body member 105 may be shaped as a tubular body, e.g. having a hollow body, and have, for example, a circular, oval or any other cross section. External elongated body member 105 may have a predetermined diameter or cross-section, e.g. in a range between 0.5 mm to 5 mm or between 1.5 mm to 3 mm. The external elongated body member 105 comprises the hollow body along the longitudinal axis 190. The hollow body is configured to have an internal diameter different from the external diameter of the internal elongated body member 130, e.g. larger than the external diameter of the internal elongated body member 130, which enables the internal elongated body member 130 to be inserted into the external elongated body member 105, and move or slide within it. It is noted that although external and internal elongated body member 105 and 130 may be cylindrical, other geometrical shapes may be devised, e.g. an elongated member with a square, rectangular, elliptical, rounded or squircle-shaped cross-section.

The penetration footprint or cross-section of the grasper may be determined according to the cross-section or diameter of the external elongated body member 105, and/or according to the cross-section or diameter of the grasping element 135.

FIG. 1C schematically illustrates the handle member 150 of the grasper 100 according to some exemplary embodiments of the subject matter. The actuating handles 107 and 108 are adapted for holding the grasper 100, e.g. by a user's single hand, for example using a finger and a thumb. The handle member 150 controls the grasping element 135 between multiple open states and a closed state, for example by pushing and/or pulling the internal elongated body member 130 through the external elongated body member 105. For example, the internal elongated body member 130 may be configured to move or slide through the external elongated body member 105, such that the movement causes opening and/or closing of the grasping element 135. The manipulation of the actuating handles 107 and 108, causes the internal elongated body member 130 to move through the external elongated body member 105, for example, the internal elongated body member 130 is inserted through the hollow body of external elongated body member 105, and slides forward and backwards through the hollow body of the external elongated body member 105.

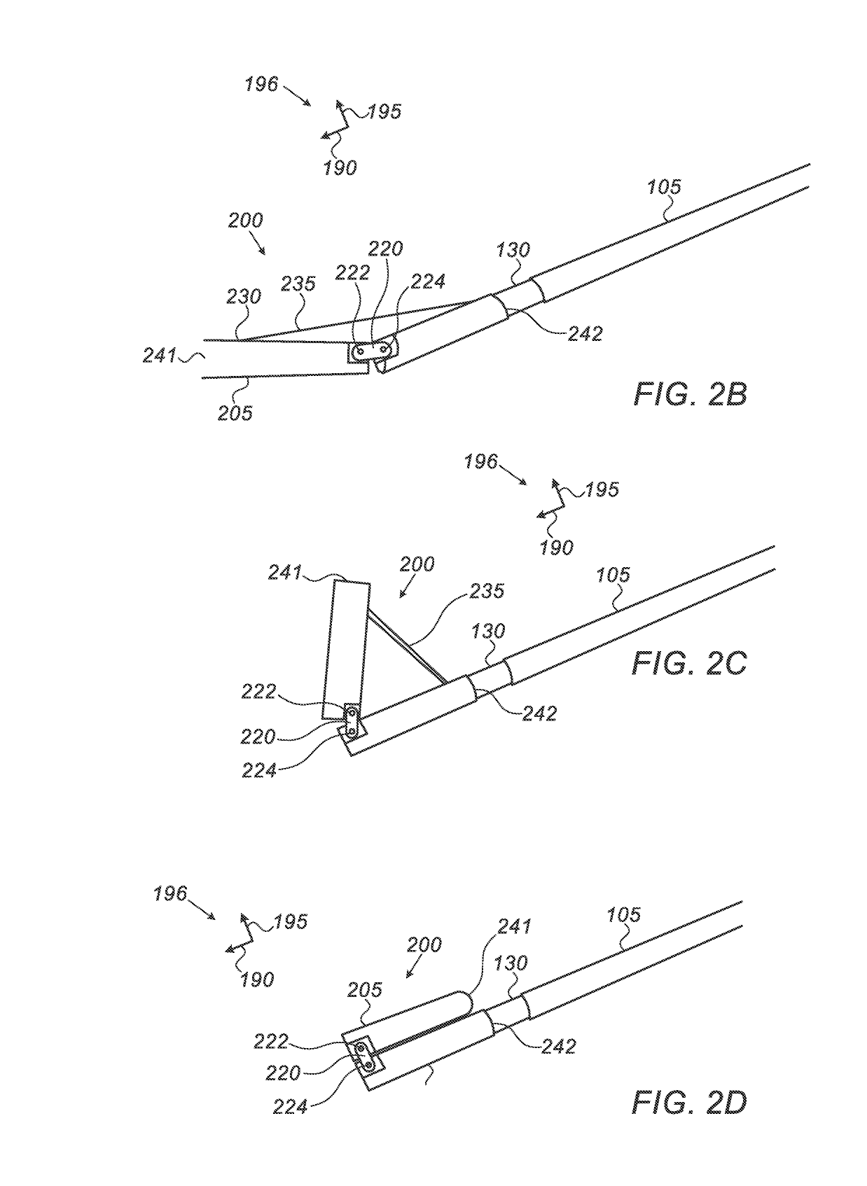

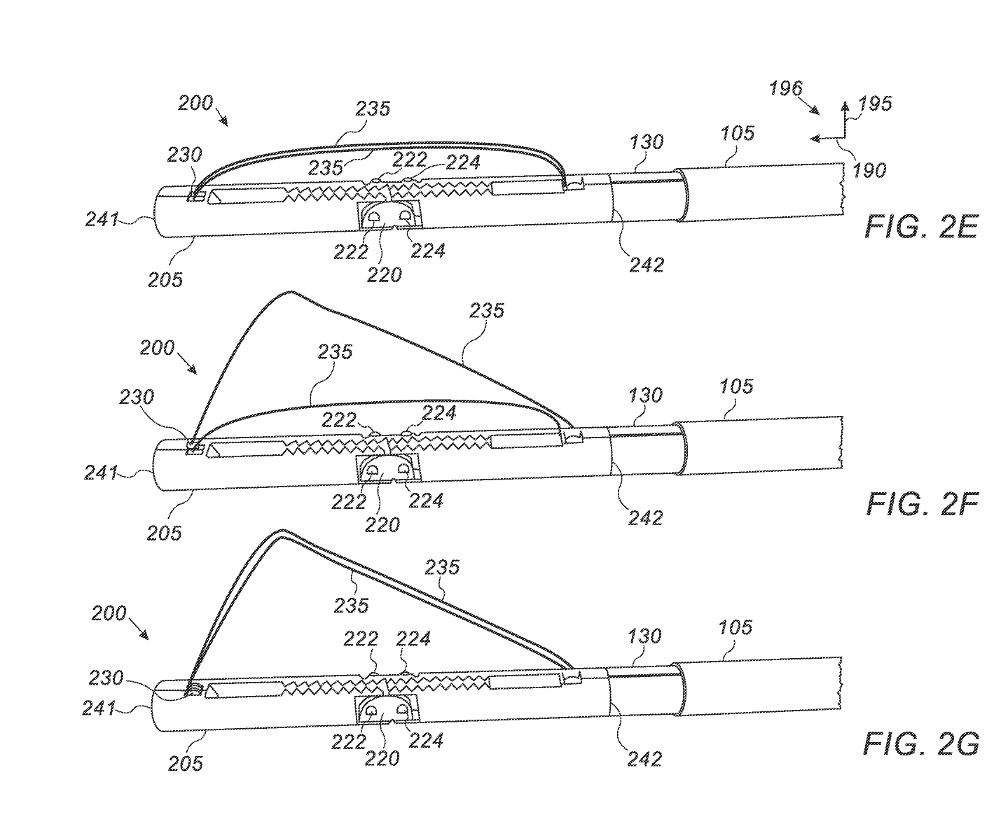

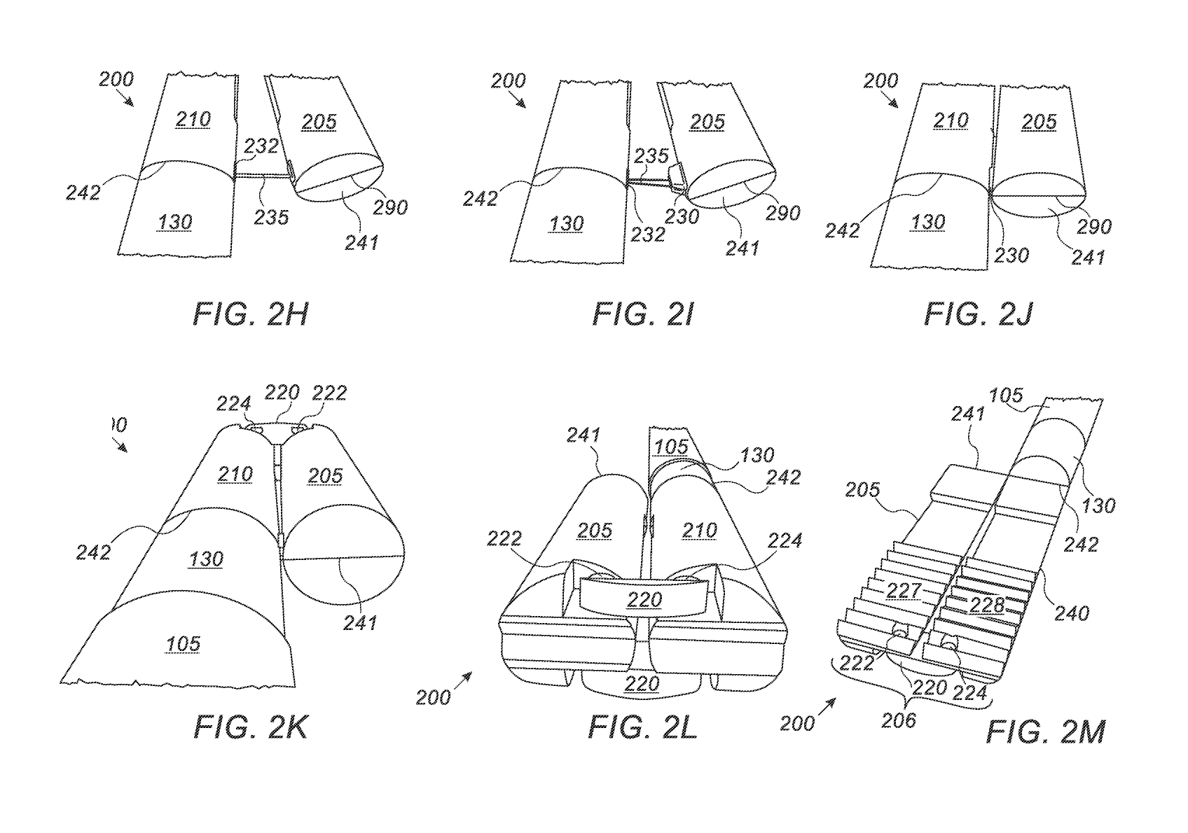

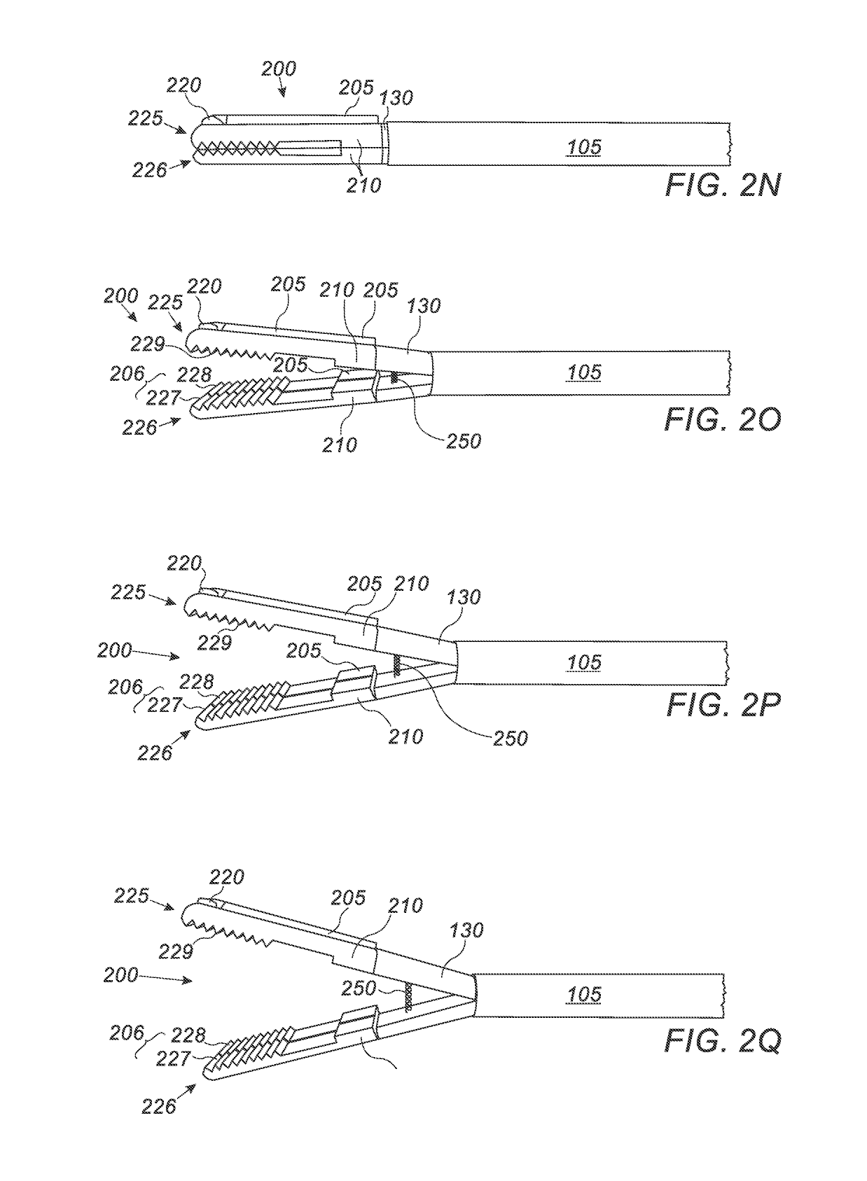

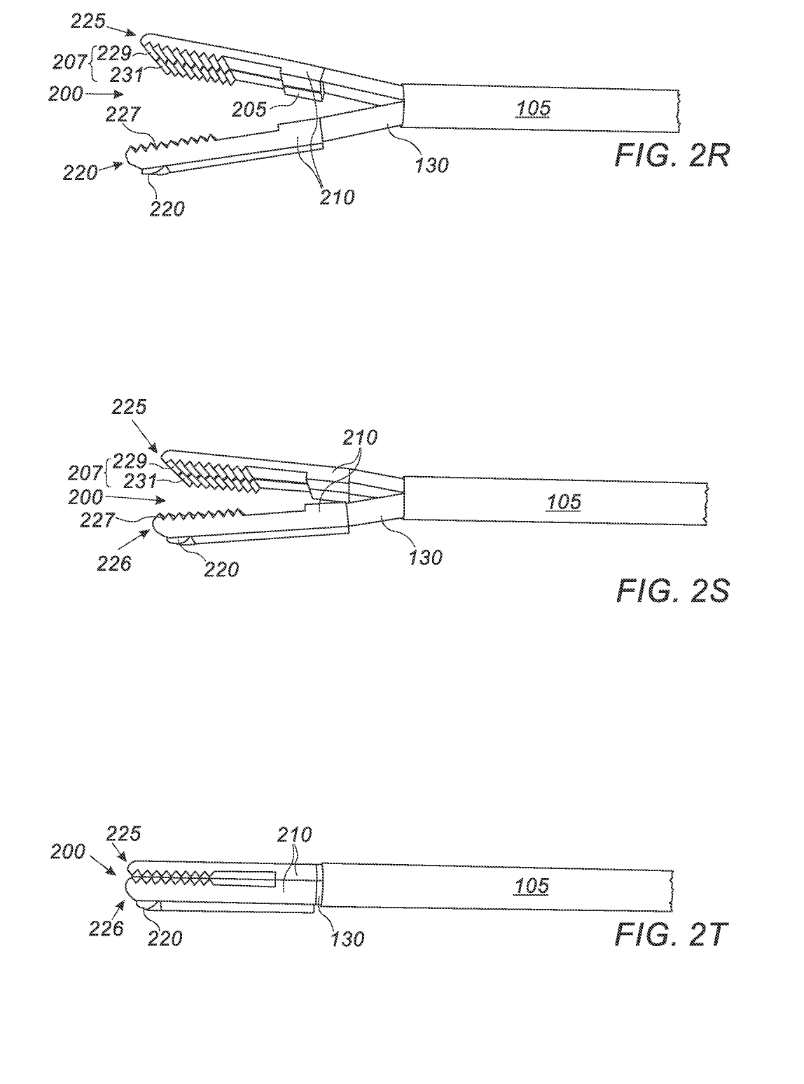

In some exemplary embodiments of the subject matter, the grasping element 135 is constructed to open and close according to the motion of the internal elongated body member 130, for example, the grasping element 135 may be a portion of the internal elongated body member 130. The grasping element 135 and the internal elongated body member 130 may be composed of a flexible material, such as flexible metals, plastics, or the like. The grasping element 135 may comprise two separably adjoining or separably connected jaws or arms, e.g. the grasping arms 136 and 137, which are connected at least at one end along the longitudinal axis 190 (e.g., at the proximal end which is closer to the proximal end of the grasper). In such an exemplary embodiment, when the grasping element 135 is moved away from the external elongated body member 105 towards the distal end 117 along the longitudinal axis 190, the distal end 117 of grasping arms 136 and 137 open away from each other. When the grasping element 135 is moved towards the external elongated body member 105 and towards the proximal end 119 along the longitudinal axis 190, the grasping arms 136 and 137 close towards each other by pivoting around the latitudinal axis 195, as shown, for example in FIGS. 1A-1B, and 2N-2T.

The movement of the grasping arms 136 and 137 is controllable via the handle member 150, which is configured to move the internal elongated body member 130 through the external elongated body member 105. The handle member 150 comprises a control mechanism handle 155, which is used to control configuration of the grasping element 135 as will be further disclosed herein, disclosing several non-limiting exemplary embodiments of configurable grasping elements providing at least two states: a narrow state having a comparatively narrow grasping surface area, and a corresponding narrow diameter or cross-section or penetration footprint of the grasping element, and an expanded state having a comparatively wider grasping surface area than the narrow grasping surface area, and a corresponding wider diameter or cross-section or penetration footprint of the grasping element.

It is noted, that in some non-limiting cases, the handle member 150 may operate the grasping element 135 mechanically as disclosed above herein, while in other non-limiting cases, the handle member 150 may additionally or alternatively operate the grasping element 135 via electrical, electronic and/or magnetic components, for example, a motor, or the like. Hence, while embodiments in the discussion herein may relate to a particular implementation for controlling and operating the grasping element 135, this should by no means be construed limiting.

FIGS. 2A-2T schematically illustrate a first exemplary embodiment of a grasping element of a grasper, according to exemplary embodiments of the disclosed subject matter. Referring to FIG. 2A, the grasping element 200 is connected to the distal end 117 of the internal elongated body member 130 of the grasper 100, and comprises a first grasping jaw portion 205 and a second grasping jaw portion 210, which are connected by a connecting member 220 that comprises rotation members 222 and 224. In the configuration in an insertion/extraction state or a narrow state of the grasping element 200 comprises a narrow cross-section, since the first grasping jaw portion 205 and the second grasping jaw portion 210 are configured adjacent to each other in a straight line along the length of longitudinal axis 190. In the narrow state, the grasping element 200 comprises a narrow cross-section, which enables safe and easy insertion or extraction of the grasping element 200 into a human or mammal body, for example, during a MIS.

The grasping element 200 comprises a control mechanism, such as a cord or a stiff or rigid element 235 which is configured for imparting traction on the jaw portion, representing any number of cords or rigid elements. The cord 235 is operably coupled to the control mechanism handle 155, which is used to control the movement function of the cord 235. It is noted that the control mechanism handle 155 may be operated manually or may be an electro-mechanical mechanism, which operates the control mechanism, e.g. the cord 235. The control mechanism, e.g. the cord 235 is operably coupled to the distal end 117 of the first grasping jaw portion 205. The cord 235 enables rotating the first grasping jaw portion 205 using the connecting member 220 in a clockwise or counterclockwise direction, relative to an axis parallel to the longitudinal axis 190 of the grasper 100. The cord 235 may also control the rotation angle of the first grasping jaw portion 205. For example, in the insertion/extraction or narrow state, the cord 235 may be at full length, e.g. no tension is applied to the cord 235 by the user of the grasper 100. The cord 235 is connected to the control mechanism handle 155, which enables the user to control the configuration of at least an insertion/extraction or narrow state and an expanded state (e.g. expansion and reduction) of the grasping element 200. It should be noted that in the insertion/extraction state or narrow state, the grasping element 200 is not intended to be used for a grasping action, but rather for a penetration or extraction maneuver of the grasping element into or from the body. It is noted, that in some exemplary embodiments of the subject matter, the control mechanism may be a stiff element imparting traction on the grasping element 200.

Referring to FIG. 2B, when tension is applied to the cord 235 by a user, for example the user pulls on the cord 235 at the handle member 150, the tension results in the cord 235 causing the first grasping jaw portion 205 to move in a clockwise direction. The cord 235 may be pulled a certain length, e.g. a third of the cord's length, depending on the amount of tension applied thereto. An angle measured between the elongated dimension of the first grasping jaw portion 205 to the elongated dimension of the second grasping jaw portion 210 may be reduced from an angle of 180 degrees to some other angle, for example an angle of sixty degrees as illustrated in FIG. 2C. As illustrated in FIG. 2D, when the cord 235 is fully pulled, the first grasping jaw portion 205 is aligned adjacent to or substantially parallel to the second grasping jaw portion 210 as further disclosed herein.

Referring to FIGS. 2B-2D, the first grasping jaw portion 205 comprises a tab 230, which is operably coupled to the cord 235 at a distal end 117 of the first grasping jaw portion 205. The cord 235 is configured to connect to the grasping element 200 from the tab 230 via grasping element cavity 232, to the proximal end 119 of the second grasping jaw portion 210. The cord 235 is threaded through grasping element cavity 232 and runs along longitudinal axis 190 through the external elongated body member 105 to the handle member 150, for example, through a cord cavity (not shown) formed within the internal elongated body member 130 until the handle member 150. The cord 235 is operably coupled to be controllable via the control mechanism handle 155, e.g. by the user of the grasper 100, to rotate the first grasping jaw portion 205 such that a grasping element distal end 241 of the first grasping jaw portion 205 is rotated towards a proximal end 242 of the second grasping jaw portion 210 as shown in FIGS. 2C-2D. The axis of rotation may be perpendicular to the longitudinal axis 190 of the grasper 100, e.g. along a plane 196.

The cross-section area 290 is shown for example in FIG. 2J. The diameter or footprint of the grasper in the narrow state is maintained to be equal to the diameter or footprint of a grasper that does not have an expanded state or configuration. Thus, the advantage of inserting a grasper via a minimal incision may be maintained, also enabling the additional benefit of allowing the grasper to assume an expanded state.

FIG. 2B shows an intermediate state of the grasping element 200, in accordance with embodiments of the disclosed subject matter. In the intermediate state, the cord is pulled by the user via the control mechanism handle 155 of grasper 100 to apply tension that causes the cords 235 to retract.

FIG. 2C shows and intermediate state of a partially retracted grasping element, in which the distal end 117 of first grasping jaw portion 205 is rotated towards the proximal end 119 of the second grasping jaw portion 210. A third and final state of rotation is shown in FIG. 2D, wherein the distal end 117 of the first grasping jaw portion 205 is adjacent to the proximal end 119 of the second grasping jaw portion 210. It is noted that during the transfer of the grasping element from a narrow state to an expanded state, the angle of rotation between first grasping jaw portion 205 to second grasping jaw portion 210 may be in the range of 0-360 degrees.

Referring to FIGS. 2E-2G, in some exemplary cases, the control mechanism includes at least one cord, represented here by two cords 235, where is each cord is operably coupled to the grasping element distal end 241 of the first grasping jaw portion 205 via the tab (also: tenon) 230, and to the proximal end 242 of the second grasping jaw portion 210, e.g. through cavity 232.

As illustrated in FIG. 2E, the grasping element 200 is in a narrow state, in which it can be inserted easily, e.g. in vivo, through a small incision. Actuation of the control mechanism handle 155 creates tension in the cords 235 which result in exposure of the tab or tabs 230, as represented herein as two tabs 230, representing any number of tabs 230, as shown in FIGS. 2F-2G. In other non-limiting exemplary embodiment, the control mechanism may comprise a push-pull lever (not shown), which controls the rotation and position of the first grasping jaw portion 205 relative to the longitudinal axis 190 of grasper 100.

Reference is made to FIGS. 2H-2L, showing the grasping element distal end 241 of the first grasping jaw portion 205 being moved towards the proximal end 242 of the second grasping jaw portion 210, according to some exemplary embodiment. Tension may be applied by a user to the cord/s 235, which in turn causes the distal end 241 of the first grasping jaw portion 205 to rotate towards the proximal end 242 of the second grasping jaw portion 210, until the grasping jaw portions are parallel, adjacent or touching. Tension may be applied to the cord 235, e.g. by a user activating the control mechanism handle 155 until the tab 230 is inserted into the grasping element cavity 232. In some cases, to ensure the second grasping jaw portion 210 is maintained in the arrangement or adjacent state of the grasping jaw portions shown in FIG. 2L, the cords 235 may be secured at a predetermined or fixed tension that maintains the grasping jaw portions in the adjacent state or configuration. The cord 235 may be secured or locked, for example, via the control mechanism handle 155, by applying a securing mechanism, such as a locking pin, ratchet, or the like.

The cord 235 may be pulled along the longitudinal axis 190, for example by moving the control mechanism handle 155 along the longitudinal axis 190 to create the necessary tension in the cord 235 to pull the first grasping jaw portion 205. When the cord 235 is in full tension the first grasping jaw portion 205 is adjacent to the second grasping jaw portion 210 such that the grasping jaw portions are both parallel to the longitudinal axis 190, thus the grasping element 200 assumes an expanded state and is ready for a grasping action or operation, e.g. grasping living tissue during a medical procedure.

FIG. 2M shows a schematic illustration of a bottom part of the grasping element 200 with a cutaway, to show only an adjoined lower grasper arm 206 of the grasping element 200 when the first grasping jaw portion 205 and the second grasping jaw portion 210 are secured in the adjacent state, according to exemplary embodiments of the subject matter. The adjoined lower grasper arm 206 comprises bottom grasping surfaces 227 and 228, which are configured to grasp tissue, where each of the bottom grasping surface 227 and 228 are a portion of the first grasping jaw portion 205 and the second grasping jaw portion 210 respectively. The grasping surfaces may be toothed, scraped and/or otherwise configured to securely grasp living tissue without causing damage to it on one hand, and without risking it slipping away on the other hand.

This exemplary embodiment includes a plurality of (narrow) bottom grasping surfaces (227, 228) configured to be adjoined, adjusted, folded and/or rotated to create a single, broader and adjoined lower grasping surface 206 of the grasping element 200. The adjoined lower grasping surface 206 created by the plurality of narrower grasping surfaces 227 and 228 provides a wider grasping surface, which increases the surface area of the grasping element 200 to grasp tissue, thus preventing or comparatively reducing the risk of the grasping element 200 damaging and/or tearing the grasped tissue. The cross-section of the grasping element 200 and of the adjoined lower grasping surface 206, comprising, for example, narrow bottom grasping surfaces (227, 228), is at least doubled, when the first grasping jaw portion 205 and the second grasping jaw portion 210 are adjacently aligned in the folded position. Thus, the cross-section of the grasping element 200 may increase, e.g. if the width of the narrow grasping surfaces (227, 228) is each within a range of 1.5 mm-3 mm, the width of the adjoined lower grasping surface 206 is within a range of 3 mm-6 mm, thus increasing the grasping surface cross-section. The expanded width of the grasping element 200 provides a larger grasping surface area, thus preventing the grasping element 200 or reducing the risk from unintentionally disconnecting or slipping away from the grasped object, e.g. tissue, or damaging the grasped object.

In some embodiments, the length of connecting member 220 may dictate the size of the grasping area, since connecting member 220 defines the distance between bottom grasping surface 227 and grasping surface 228 when the first grasping jaw portion 205 and the second grasping jaw portion 210 are adjacently aligned in folded position (see for example FIGS. 2L and 2M). Thus, the size of connecting member 220 may be considered to be an important factor in determining the grasping surface cross-section. That is, the longer connecting member 220 is, the larger can be the grasping surface cross-section, and the shorter connecting member 220 is, the smaller the grasping surface area.

In some exemplary embodiments of the subject matter, the grasping element 200 may comprise a plurality of grasping jaw portions, such that the plurality of grasping jaw portions are configured to be positioned along the length of a longitudinal axis 190 of the grasper, e.g., wherein the elongated dimension of the first grasping jaw portion 205 is arranged substantially along a straight line in relation to the second grasping jaw portion 210 (and possibly to additional grasping jaw portions).

The plurality of grasping jaw portions may be coupled together by connecting members, e.g. similar to connecting member 220 that connects the first grasping jaw portion 205 and the second grasping jaw portion 210. The plurality of grasping jaw portions may rotate in a manner similar to the rotation of the first grasping jaw portion 205 in relation to the second grasping jaw portion 210 as described herein above.

FIG. 2N-2T show the grasping element 200 for use during an in vivo MIS or operating procedure, according to some exemplary embodiments of the subject matter. The grasping element 200 comprises bottom grasping surfaces 227, 228, and top grasping surfaces 229, 231, which provide two enlarged surface grasping surfaces for grasping, for example, in vivo tissue during the MIS. In some non-limiting exemplary embodiments of the subject matter, the grasping element 200 is coupled to the internal elongated body member 130. The configuration of the grasping element 200 as shown in FIGS. 2N-2T represents the expanded state of the grasping element 200, in which the grasping element 200 comprises an expanded cross-section enabling the grasping operation, e.g. grasping living tissue, during a medical procedure.

When the surgical portion of the procedure is completed, and the grasping element 200 is no longer used for grasping in vivo tissue, the grasping element is reconfigured to the insertion/extraction or narrow state, as shown in FIG. 2A, in order to allow safe extraction of the grasper 100 from the human or mammal body while it is adjusted back to its minimal width or cross-section.

It is noted that while certain embodiments may only be described with respect to certain figures or elements (e.g., grasping element 200), this should by no means be construed limiting or imply that these embodiments may not be equally or correspondingly applicable to other figures or elements (e.g., grasping elements 400 and 600). For instance, embodiments referring to the opening and closing of grasping element 200 may be analogously applicable to grasper elements 400 and 600.

According to some embodiments, grasping element 200 is configured to be slidable through and outside the distal end of external elongated body member 105. The more grasping element 200 protrudes out of the distal end of external elongated body member 105, the more grasping element 200 may open, i.e., the distance between top grasping element 225 and bottom grasping element 226 may increase. When the entirety of grasping element 200 is outside external elongated body member 105, grasping element 200 may assume a fully open and expanded state (see for example FIGS. 2Q and 2R). In other words, the more grasping element 200 and internal elongated body member 130 (which are connected with each other) are pushed out of external elongated body member 105 via the distal end thereof, the more grasping element 200 may open, e.g., the more distant top grasping element 225 may become from bottom grasping element 226, optionally until grasping element 200 reaches its most expanded rest state. Contrariwise, the more grasping element 200 and internal elongated body member 130 are pulled back into external elongated body member 105 via the distal end, the lesser grasping element 200 is open, i.e., top grasping element 225 and bottom grasping element 226 may come closer to each other, optionally reconfiguring grasping element 200 into its contracted narrow state in order to allow safe extraction of grasper 100 from the human or mammal body via the proximal end (not shown) of external elongated body member 105. Top grasping element 225 and bottom grasping element 226 may be urged by external elongated body member 105 to come closer or approach each other, as grasping element 200 is pulled back into external elongated body member 105. That is, grasping element 200 closes when it is pulled back into external elongated body member 105.

In some embodiments, grasping element 200 may automatically open while being and/or after it/they are pushed out of external elongate body member 105.

In some embodiments, at least a portion of grasping element 200 may be made of elastically deformable material, shape memory material (e.g., shape memory alloy or a shape memory polymer), or any other material or composition of material capable of returning to its original shape after it is deformed.

According to some embodiments, grasping element 200 is configured to have an original open state, which is gradually elastically “deformed” into a comparatively more closed state, when grasping element 200 is pulled back into the distal end of external elongated body member 105. Contrariwise, when grasping element 200 is pushed outside of the distal end of external elongated body member 105, grasping element 200 may (gradually) open, optionally until it assumes its original fully open state.

As already indicated, herein, grasping element 200 may be made, at least partially, of shape memory material, and configured so that grasping element 200 may alter from a closed state to an open state upon an external trigger. For example, responsive to subjecting the grasping element 200 to a characteristic change in temperature or the cessation of external force, grasping element 200 may open from a closed shape to an original, open state.

In some embodiments, as illustrated for example in FIGS. 2O-2Q, a mechanical energy storage device 250 such as, for example, a spring-like element, a pneumatic arrangement and/or the like, may be positioned between the two units of internal elongated body member 130 in order to assist in or cause automatic opening of grasping element 200, e.g., as it is pushed out of the distal end of external elongated body member 105. For example, grasping element 200 may be urged to open by a mechanical energy storage device 250 such as, for example, a spring-like element. The mechanical energy storage device 250 may, for example, impart an expanding force on internal elongated body member 130, which in turn may impart an opening force between top grasping element 225 and bottom grasping element 226, the opening force pushing these elements away from each other and increasing the distance between top grasping element 225 and bottom grasping element 226, thus allowing grasping element 200 to expand.

In some embodiments, the mechanical energy storage device 250 may be selectively actuatable. Accordingly, a user may determine if and when to actuate the mechanical energy storage device 250 such that the mechanical energy storage device 250 would impart an opening force on the two units of internal elongated body member 130, which would push top grasping element 225 away from bottom grasping element 226 thereby causing grasping element 200 to open.

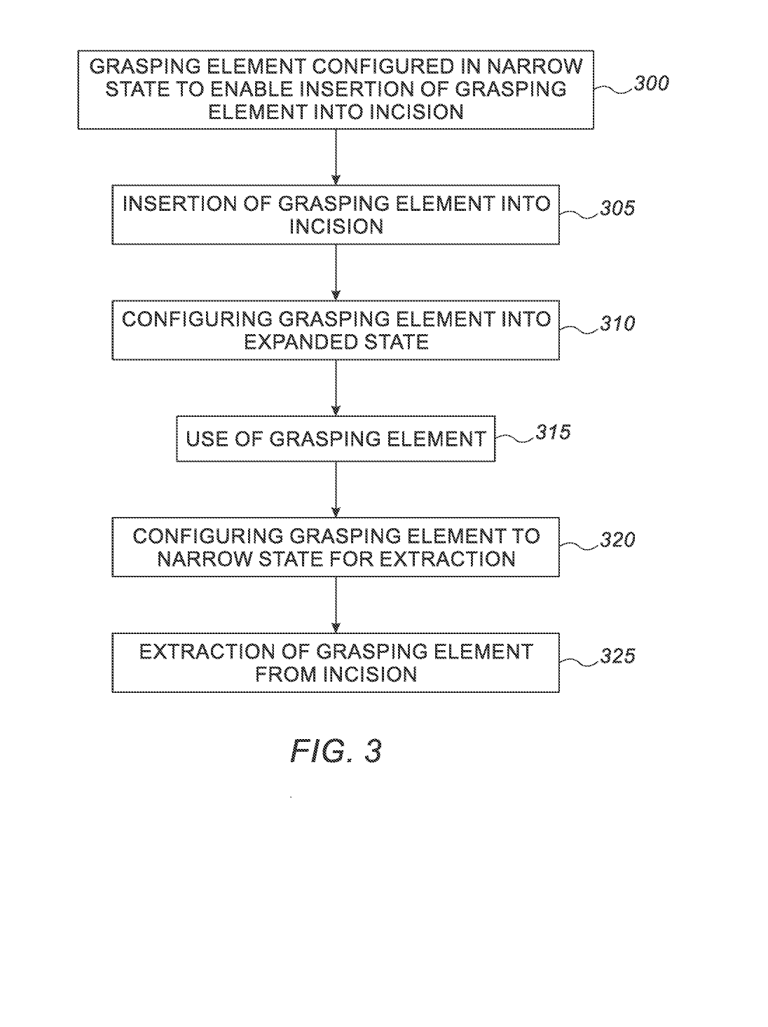

FIG. 3 is a schematic illustration of a flow chart illustrating operation of a grasper, according to some exemplary embodiments of the subject matter. Step 300 discloses the grasping element 200 configured in a narrow state to enable insertion of the grasping element into an incision. In the narrow state, the grasping element 200 is configured to be narrow, for example as disclosed herein regarding FIGS. 2A, 4A, 4D, 5A, 5B, 5D and/or 6A.

Step 305 discloses insertion of the grasping element 200 into an incision or body lumen opening.

Step 310 includes configuring the grasping element 200 to an expanded/widened grasping state. The control mechanism, e.g. the cord 235, is actuated by the user via the control mechanism handle 155 causing the grasping surface of grasping element 200 to expand. The expansion of the grasping surface of grasping element 200 as described herein provides an expanded grasping surface for grasping tissue without causing damage or tearing to in vivo tissue during the medical procedure. It is noted that in some cases, the control mechanism handle 155 comprises an electronic system, e.g. a motor, which, when activated, actuates and operates the control mechanism, e.g. the cord 235 to expand or retract the grasping element 200.

In some cases, the grasping element 200 may be arranged in an intermediate configuration state as disclosed in FIG. 2C when the cord 235 is pulled until the angle 239 between first grasping jaw portion 205 to second grasping jaw portion 210 is sixty degrees. It should be noted that such intermediate configurations are intermediate steps taken to configuring the grasping element 200 to the expanded state.

Step 310 includes using the grasping element 200 for grasping, e.g. grasping in vivo living tissue during a medical procedure. During the medical procedure the grasping element 200 is used to grasp in vivo tissue using the expanded grasping surface.

After the tissue is released the grasping element 200 is configured to back to the narrow state for extraction from the body, in Step 315. The user of the grasper 100 actuates the control mechanism handle 155 to reduce the cross-section of the grasping element 200, e.g. by reconfiguring the relative position of the first and second grasping jaw portions 205 and 210, to enable extraction of the grasping element 200 through a comparatively narrow or minimal incision, without requiring enlargement of the incision, thereby allowing in vivo operation using an expanded grasping surface of the grasping element 200, while maintaining a narrow penetration incision to reduce recovery time. Alternatively or additionally, the grasper may be used to penetrate body lumens, e.g. through natural orifices, achieving the same result of an expanded grasping surface while operating in vivo, and having a narrow insertion diameter, cross-section, penetration footprint or profile.

Step 320 discloses extraction of the grasping element 200 from the incision. Once the grasping element 200 is configured back to its narrow state, the grasping element is extracted from the human or mammal body, e.g. via the incision or the natural orifice.

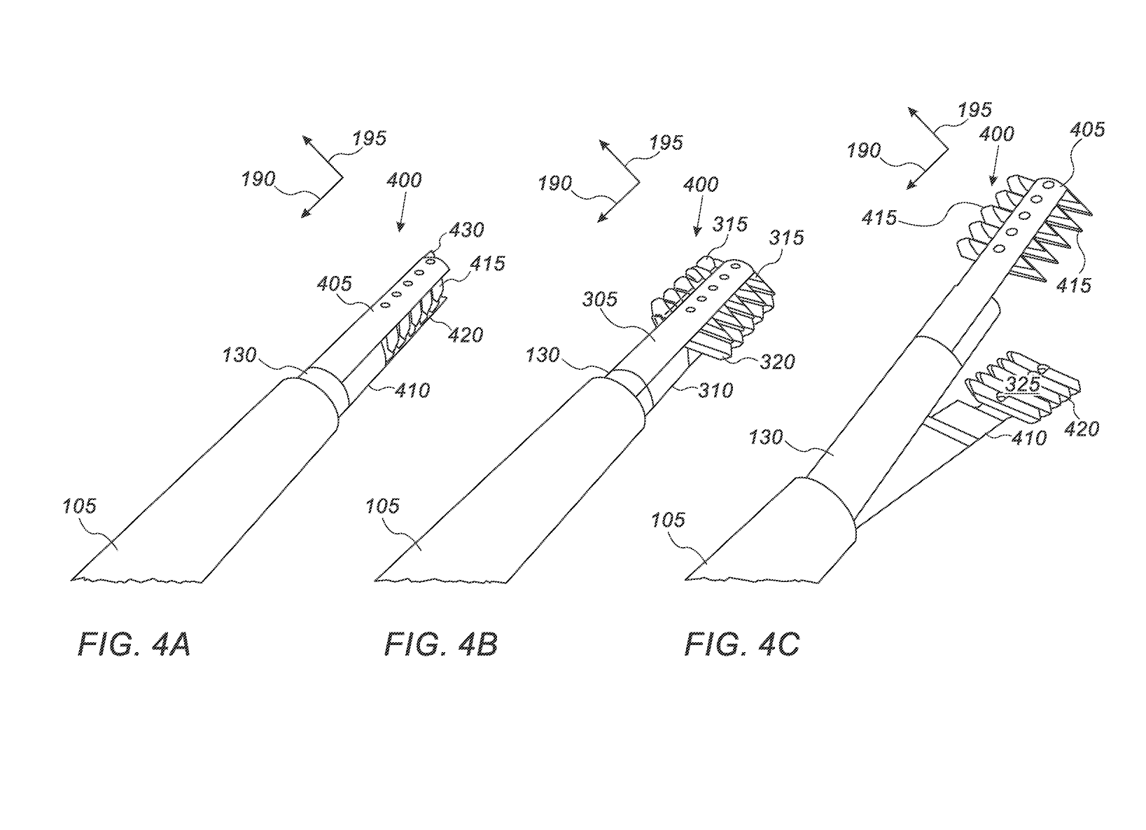

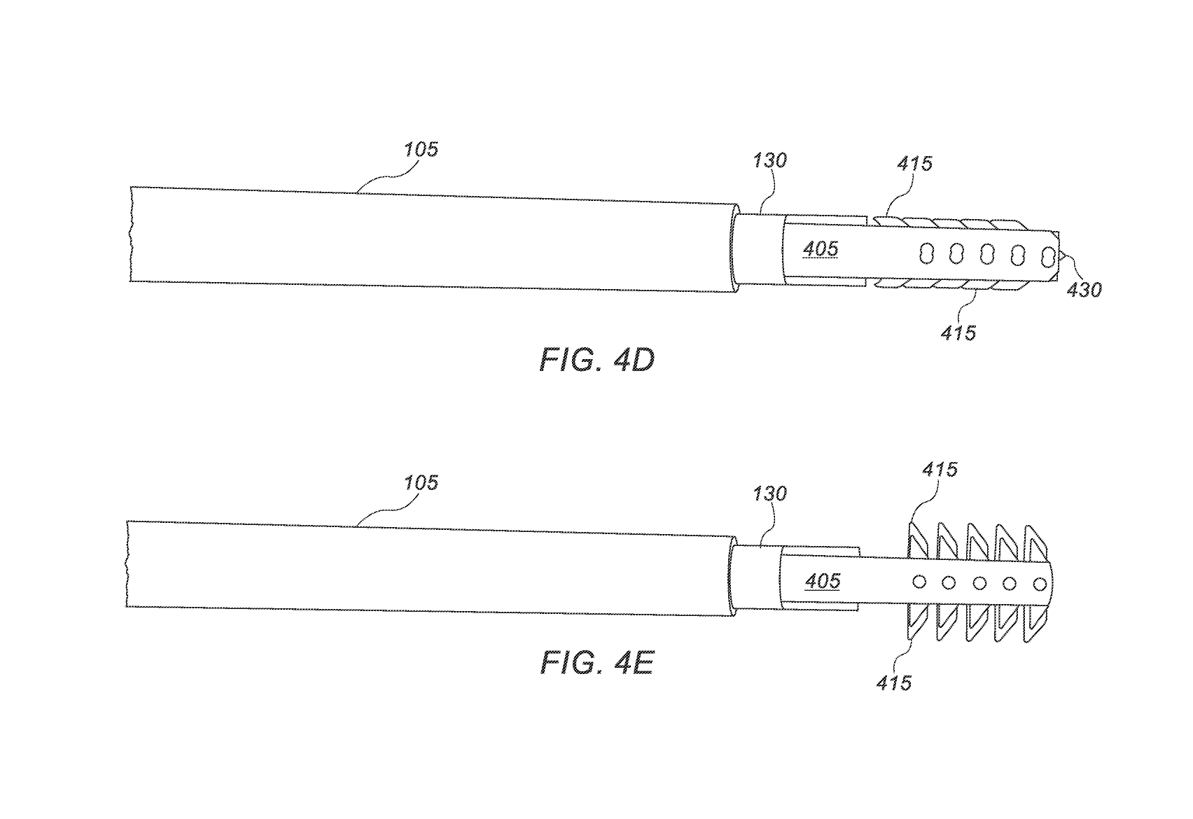

FIGS. 4A-4E schematically illustrate a second exemplary embodiment of a grasping element of a grasper, according to exemplary embodiments of the disclosed subject matter. Referring to FIG. 4A, the grasping element 400 comprises a first grasping arm 405 and a second grasping arm 410. Each grasping arm of the first grasping arm 305 and the second grasping arm 410 comprises grasping ridges 415 and 420, representing any number grasping ridges. In a narrow or insertion/extraction state, the grasping ridges 415, 420 are configured to a retracted state in which the grasping ridges 415, 420 are retracted to enable insertion of the grasping element 400 in to the human or mammal body with a minimal diameter of the incision required.

In some non-limiting embodiments, the grasping element 400 may comprise a puncturing element 430, which enables use of the grasping element to create the incision for insertion of the grasping element 400 during the medical procedure. The puncturing element 430 is beneficial, allowing a medical professional to use a reduced number of tools for creating the incision opening for the grasping element 400 to be inserted. The grasping element 400 is inserted with the grasping ridges 415, 420 retracted, e.g. the grasping element is configured to a narrow state having an initial narrow diameter, cross-section, or penetration footprint.

FIG. 4B schematically illustrates the grasping element 400 configured to assume a grasping state or expanded state, having an expanded cross-section, according to exemplary embodiments of the subject matter. Once the grasping element 400 is inserted into the incision opening and is inside the body, the grasping element 400 may be configured to the expanded state while the grasping element is in vivo.

To achieve the expanded state, the grasping ridges 415, 420 may be extended outwardly from the first and second grasping arms 405, 410 in a perpendicular direction, relative to the longitudinal axis 190 of the grasping element 400. The grasping ridges 415, 420 may be extended or retracted via control mechanism handle 155, which controls the grasping ridges via a control mechanism (not shown), e.g. cord, push/pull mechanism or the like. For example, when the control mechanism handle 155 is pushed towards the distal end 117 by the user of the grasping element 400 along a longitudinal axis 190, the ridges 415 and 420 are expanded outwardly from the grasping arms 405 and 410, the expansion being in a direction perpendicular to the longitudinal axis 190. Thus, the grasping element 400 assumes an expanded state, in which the width of grasping surface is expanded relative to the initially narrow insertion width.

When the control mechanism handle 155 is pulled towards the proximal end 119 by the user of the grasping element 400 along a longitudinal axis 190 the ridges 415 and 420 are retracted, and thus the grasping element 400 is configured back to the narrow state, which enables safe extraction of the grasping element 400 via a narrow incision or a narrow orifice when the grasping element 400 is no longer required to be grasping tissue.

FIG. 4C schematically illustrates the grasping element 400 in the expanded state with the grasping arms 405 and 410 separated to enable grasping, according to exemplary embodiment of the subject matter.

FIG. 4D schematically illustrates from a top view the grasping element 400 in the narrow state, according to exemplary embodiments of the subject matter.

FIG. 4E schematically illustrates from a top view the grasping element 400 in the expanded state, according to exemplary embodiments of the subject matter.

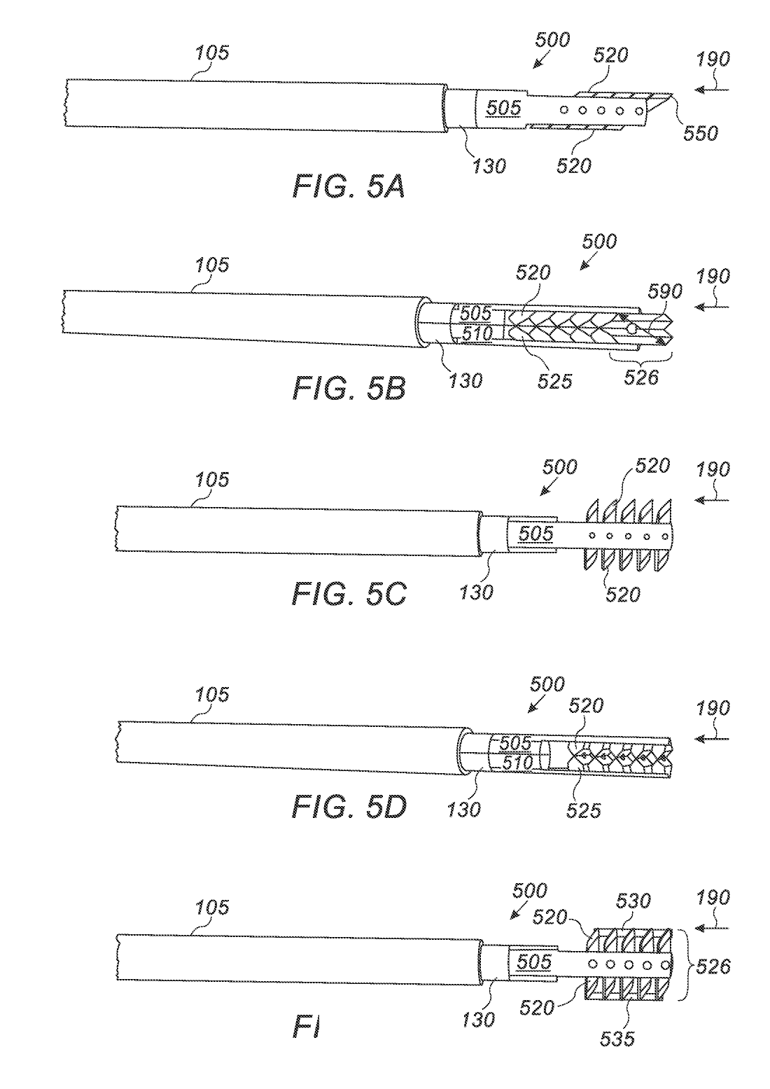

FIGS. 5A-5D schematically illustrate a third exemplary embodiment of a grasping element of a grasper, according to exemplary embodiments of the disclosed subject matter.

FIG. 5A schematically illustrates a top view of the grasping element 500, according to exemplary embodiments. The grasping element 500 comprises a top grasping arm 505, a plurality of top ridges 520 and a puncturing element 550. The puncturing element 550 enables use of the grasping element to create the incision for insertion of the grasping element 400 during the medical procedure.

Referring to FIG. 5B, the grasping element 500 comprises grasping arms SOS and 510. The grasping element 500 comprises a plurality of top ridges 520 and a plurality of bottom blades, ridges or edges 525, which are extractable or expandable or protrudable to provide a broader grasping surface area 526. The plurality of ridges 520 and 525 may be configured as blades or ridges or teeth. The grasping ridges 520, 525 (also: ridges) may be extended or retracted via cords (not shown) controlled by a user via the control mechanism handle 155. For example, when the control mechanism handle 155 is pushed towards the distal end 117 by the user of the grasping element 500 along a longitudinal axis 190 the grasping ridges 520, 525 expand along a direction, which is perpendicular to the longitudinal axis 190. Thus, the grasping element is in the expanded state. When the control mechanism handle 155 is pulled towards the proximal end 119 by the user of the grasping element 500 along a longitudinal axis 190 the grasping ridges 520 and 525 are retracted along the direction, which is perpendicular to longitudinal axis 190. Thus returning the grasping element 500 to the narrow state, which enables extraction of the grasping element 500 when the grasping element 500 is no longer in use.

FIG. 5D schematically illustrates the grasping element 500 in the narrow state, according to some exemplary embodiments of the subject matter. The grasping element 500 is configured in the narrow state to enable insertion of the grasping element 500 into as narrow as possible an incision to minimize the size of the opening required to insert the grasping element 500.

FIG. 5E schematically illustrates the grasping element 500 in the expanded state having the expanded cross-section, according to some exemplary embodiments of the subject matter. The grasping ridges 520, 525 are expanded to provide the extended width of surface area 526 and expanded cross-section. In some cases, where the grasping ridges 520, 525 are composed of hard materials that may cause damage due to sharp edges, the grasping ridges 520, 525 may comprise edge covers 530 and 535. The edge covers 530 and 535 may further ensure that all grasping ridges 520, 525 open in unison and that no grasping ridge of the grasping ridges 520, 525 does not extend.

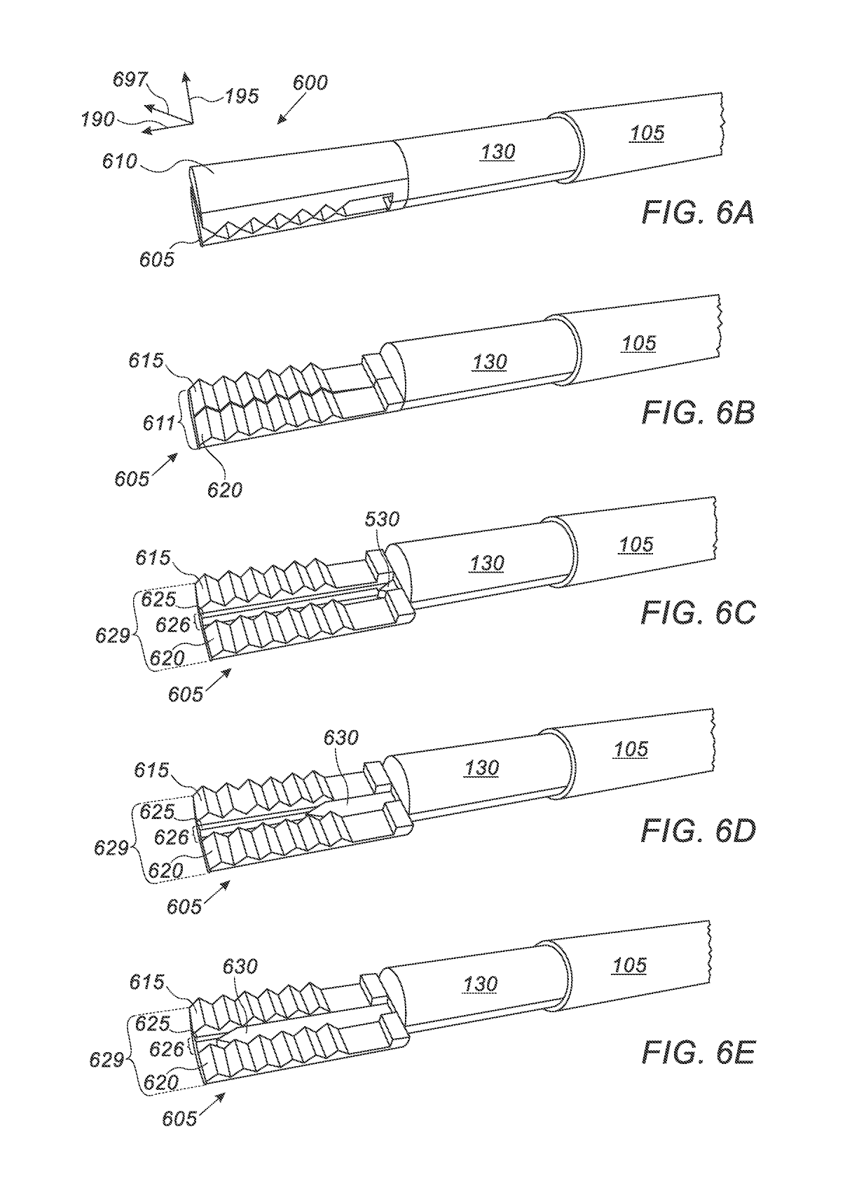

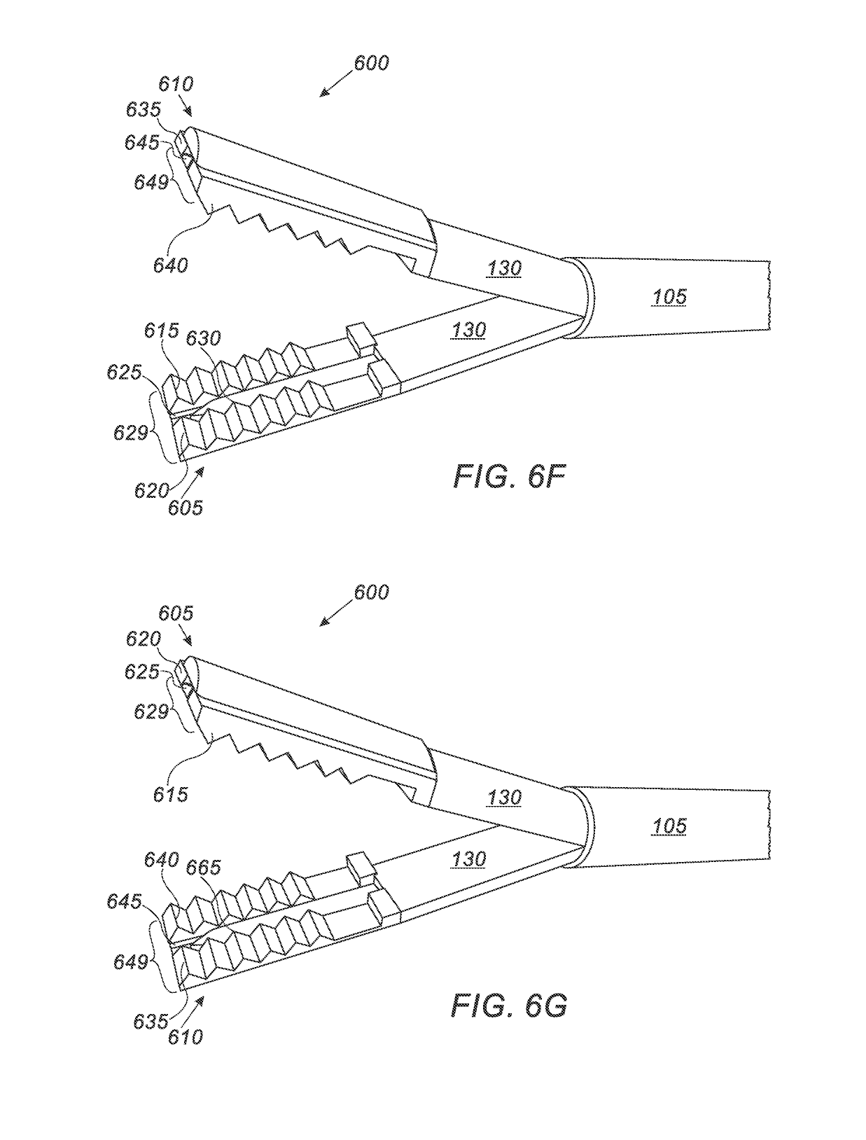

FIGS. 6A-6F schematically illustrate a fourth exemplary embodiment of a grasping element of a grasper, according to exemplary embodiments of the disclosed subject matter. FIG. 6A schematically illustrates the grasping element 600, according to some exemplary embodiments of the subject matter. The grasper 100 comprises a grasping element 600, which is used to grasp tissue, for example, during an MIS. Grasping element 600 comprises a bottom grasping arm 605 and a top grasping arm 610. The grasping element 600 is connected to the internal elongated body member 130. The grasping element 600 disclosed in FIG. 6A is in an insertion/extraction state, which enables insertion of the grasping element 600 through a small incision, for example, the required incision size is the diameter or cross-section width of the external elongated body member 105, which is inserted in vivo.

Reference is made to FIGS. 6B-6E that schematically illustrate a cutaway of the grasping element 600 to show only the bottom grasping arm 605, according to some exemplary embodiments of the subject matter. FIG. 6B schematically shows the bottom grasping arm 605, according to exemplary embodiments. The bottom grasping arm 605 comprises a first bottom grasping surface 615 and a second bottom grasping surface 620, which make up the narrow width of grasping surface 611 of grasping arm 605, when the grasping element 600 is configured to a narrow state. In the narrow state, the grasping element 600 has a narrow cross-section or penetration footprint.

Referring to FIG. 6C, the distance between the first bottom grasping surface 615 and the second bottom grasping surface 620 is adjustable or configurable, according to embodiments of the disclosed subject matter. The bottom grasping arm 605 comprises a bottom channel or cavity 625, into which a control mechanism, e.g. a bottom wedge 630 is inserted, e.g. substantially along the plane of the grasping surface, to expand the grasping surface 611 of the bottom grasping arm 605. The distance 626 between first bottom grasping surface 615 and second bottom grasping surface 620 may be increased in order to obtain a broader or expanded grasping surface 629 In one embodiment, the control mechanism, e.g. the bottom wedge 630 may be inserted into the bottom grasping element cavity 625 to increase the width of grasping surface 629 of the bottom grasping arm 605. The expanded width of the grasping surface 629 further expands the cross-section of the grasping element 600 The bottom wedge 630 is inserted and extracted from the bottom cavity 625 via the control mechanism handle 155. For example, pushing on the control mechanism handle 155 along the longitudinal axis 190 inserts the bottom wedge 630 into the bottom cavity 625 and pulling the control mechanism handle 155 along the longitudinal axis 190 extracts the bottom wedge 630 out of the bottom cavity 625. The first bottom grasping surface 615 and second bottom grasping surface 620 may be moveable along an axis 697 that is perpendicular to the longitudinal axis 190 and the latitudinal axis 195 of the grasping element 600. For example, the grasping surfaces 615, 620 may be slidable along one or more rails or bars that may be disposed perpendicularly to longitudinal axis 190 and to latitudinal axis 195. Sliding the first bottom grasping surface 615 and second bottom grasping surface 620 may be performed using the control mechanism handle 155 to insert the bottom wedge 630 into the bottom cavity 625.

FIG. 6D schematically illustrates the bottom wedge 630 being inserted partially into the bottom cavity 625, for example half the length of the bottom cavity along the longitudinal axis 190, according to exemplary embodiments.

FIG. 6E schematically illustrates the bottom wedge 630 being inserted completely into the bottom cavity 625, for example half the length of the bottom cavity along the longitudinal axis 190, according to exemplary embodiments.

It should be noted that although the cutaway Figures, e.g. FIGS. 6A-6E, show only the bottom grasping arm 605, however the description described herein regarding the bottom grasping arm 605 may describe the function and performance of the top grasping arm 610 in a similar or identical manner.

Reference is made to FIG. 6F, which shows the grasping element 600 configured in the operational state with the increased grasping surfaces, according exemplary embodiments of the subject matter. The grasping element 600 comprises the top grasping arm 610, which comprises top grasping surfaces 635 and 655, and a top channel or cavity 645. The grasping element 600 comprises the bottom grasping arm 605, which comprises bottom grasping surfaces 615, and 620, the bottom channel or cavity 625 and the bottom wedge 630.

The grasping element 600 comprises extended grasping surfaces 629 and 649. The extended grasping surfaces 629 and 649 provide a wider grasping surface, for example, if the width of grasping surfaces 615 and 620 is in the range of 1 mm-2 mm, the increased width of the grasping surface 629 may be within a range of 2.5 mm-6 mm, which includes the width of the bottom wedge 630, e.g. a range of 0.5 mm-1.5 mm. In some exemplary embodiments of the subject matter, the expansion of grasping arms 605 and 610 may be in a diagonal direction. Complete insertion of the bottom wedge 630 and the top wedge 665 expands the grasper arms 605 and 610, which is an extended or expanded state of the grasping element 600, in which the grasping element 600 is used for grasping objects, e.g. in vivo tissue.

It would be appreciated by one skilled in the art, that to return the grasping element 600 from the insertion/extraction state to the operational or extended state, the wedges 630, 665 are removed from the cavities 625, 645 via the control mechanism handle 155, and are inserted, for example, into the internal elongated body member 130. For example, where the control mechanism handle 155 is a push/pull mechanism, pushing the control mechanism handle 155 towards the distal end 117 along the longitudinal axis 190 results in the wedges 630 and 665 being inserted into the cavities 625 and 645 and expansion of the grasping element 600. Conversely, pulling the control mechanism handle 155 towards the distal end 117 along the longitudinal axis 190 results in the wedges 630 and 665 being extracted from the cavities 625 and 645 and narrowing of the grasping element 600.

In some exemplary embodiments of the subject matter, the grasping element 600 may be configured to comprise a hinge or pivot, which enables configuring the grasping arms 605 and 610 in a plurality of intermediate expansion states. The plurality of intermediate expansion states is achieved according to the distance at which the wedges 630, 665 are inserted into cavities 625, 645. The grasping arms 605 and 610 are attached to the pivot, such that the insertion of the wedges 630 and 665 into the cavities 625 and 645 results in the grasping arms 605 and 610 pivoting away from each other along the longitudinal axis 190. For example, where the wedges 630 and 665 are inserted into the cavities 625 and 645 respectively, for half the length of the cavities 625 and 645, the grasping element 600 may expand half of the maximum possible expansion width of the grasping element 600.

It is noted that in the embodiments described herein, when the grasper is configured to the narrow state, the diameter or cross-section of the grasping element 200 does not increase as a result of the expansion capability or option of the grasping elements 200 of FIG. 2, 400 of FIG. 4, 500 of FIG. 5, and 600 of FIG. 6. Rather, the grasping elements 200, 400, 500, and 600 maintain a minimal cross-section when the grasper is configured to the narrow state. The structural elements that allow the expansion of the grasping element surface area, such as wedge 630 of FIG. 6, connection member 220, rotation members 222 and 224 of FIG. 2, the grasping ridges 415, 420 of FIG. 4, and/or grasping ridges 520 and 525 of FIG. 5, enable the grasper to obtain an expanded configuration, while the narrow state is maintained within the original narrow diameter or cross-section of the grasper, e.g. the narrow state configuration of the grasping element 200, 400, 500, and 600 and/or the external elongated body member 105, thus allow maintaining a narrow penetration footprint as well as an expanded grasping work mode/state.

It is noted that in some exemplary embodiments of the subject matter, the control mechanism handle 155 may operate manually to expand or retract the grasping elements 200, 400, 500, 600. In other exemplary embodiments of the subject matter, the control mechanism handle 155 may operate via an electronic component, e.g. a motor, to expand or retract the grasping elements 200, 400, 500, 600.

The corresponding structures, materials, acts, and equivalents of all means or step plus function elements in the claims below are intended to include any structure, material, or act for performing the function in combination with other claimed elements as specifically claimed. As used herein, the singular forms “a”, “an” and “the” are intended to include the plural forms as well, unless the context clearly indicates otherwise. It will be further understood that the terms “comprises” and/or “comprising” and/or “having” when used in this specification, specify the presence of stated features, integers, steps, operations, elements, and/or components, but do not preclude the presence or addition of one or more other features, integers, steps, operations, elements, components, and/or groups thereof.

Unless otherwise indicated or applicable, the word “or” in the description and claims is considered to be the inclusive “or” rather than the exclusive or, and indicates at least one of, or any combination of items it conjoins.

As used herein the term “configuring” and/or ‘adapting’ for an objective, or a variation thereof, implies using materials and/or components in a manner designed for and/or implemented and/or operable or operative to achieve the objective.

Unless otherwise specified, the terms ‘about’ and/or ‘close’ with respect to a magnitude or a numerical value may imply to be within an inclusive range of −10% to +10% of the respective magnitude or value.

Unless otherwise specified, the terms ‘about’ or ‘close’ imply at or in a region of, or close to a location or a part of an object relative to other parts or regions of the object.

In the discussion, unless otherwise stated, adjectives such as “substantially” that modify a condition or relationship characteristic of a feature or features of an embodiment of the invention, are to be understood to mean that the condition or characteristic is defined to within tolerances that are acceptable for operation of the embodiment for an application for which it is intended.

When a range of values is recited, it is merely for convenience or brevity and includes all the possible sub-ranges as well as individual numerical values within and about the boundary of that range. Any numeric value, unless otherwise specified, includes also practical close values enabling an embodiment or a method, and integral values do not exclude fractional values. A sub-range values and practical close values should be considered as specifically disclosed values.

As used herein, ellipsis ( . . . ) between two entities or values denotes an inclusive range of entities or values, respectively. For example, A . . . Z implies all the letters from A to Z, inclusively.

Positional terms such as “upper”, “lower” “right”, “left”, “bottom”, “below”, “lowered”, “low”, “top”, “above”, “elevated”, “high”, “vertical” and “horizontal” as well as grammatical variations thereof as may be used herein do not necessarily indicate that, for example, a “bottom” component is below a “top” component, or that a component that is “below” is indeed “below” another component or that a component that is “above” is indeed “above” another component as such directions, components or both may be flipped, rotated, moved in space, placed in a diagonal orientation or position, placed horizontally or vertically, or similarly modified. Accordingly, it will be appreciated that the terms “bottom”, “below”, “top” and “above” may be used herein for exemplary purposes only, to illustrate the relative positioning or placement of certain components, to indicate a first and a second component or to do both.

The terminology used herein should not be understood as limiting, unless otherwise specified, and is for the purpose of describing particular embodiments only and is not intended to be limiting of the disclosed subject matter. While certain embodiments of the disclosed subject matter have been illustrated and described, it will be clear that the disclosure is not limited to the embodiments described herein. Numerous modifications, changes, variations, substitutions and equivalents are not precluded.

Terms in the claims that follow should be interpreted, without limiting, as characterized or described in the specification.

It is appreciated that certain features of the invention, which are, for clarity, described in the context of separate embodiments or example, may also be provided in combination in a single embodiment. Conversely, various features of the invention, which are, for brevity, described in the context of a single embodiment, example and/or option, may also be provided separately or in any suitable sub-combination or as suitable in any other described embodiment, example or option of the invention. Certain features described in the context of various embodiments, examples and/or options are not to be considered essential features of those embodiments, unless the embodiment, example and/or option is inoperative without those elements.

1. A grasper, comprising:
a grasping element at a distal end of the grasper;
wherein the grasping element is configurable to at least a narrow state having a narrow cross-section, and at least an expanded state having an expanded cross-section;
wherein the grasping element configured in the narrow state is insertable through an incision or narrow orifice into a body;
wherein the grasping element configured in the expanded state has an increased grasping surface; and
wherein the grasping element is configurable from the narrow state to the expanded state after insertion into the body.

2. The grasper of claim 1, wherein the grasping element is configurable from the expanded state back to the narrow state prior to extraction of the grasping element from the body.

3. The grasper of claim 1, wherein the grasping element comprises:
a first grasping jaw portion;
a second grasping jaw portion;
a connecting member coupling the first grasping jaw portion and the second grasping jaw portion; and
a control mechanism operably coupled to the distal end of the first grasping jaw portion; wherein the first grasping jaw portion rotates around the connecting member when the control mechanism is activated; wherein the rotation causes a distal end of the first grasping jaw portion to pivot around an axis that is perpendicular to a longitudinal axis of the grasper, such that the distal end of the first grasping jaw portion becomes, after completing the rotation, adjacent to a proximal end of the second grasping jaw portion; and
a grasping surface comprising grasping ridges of the first grasping jaw portion adjacent to grasping ridges of the second grasping jaw portion.

4. The grasper of claim 3, further comprising:
a handle member at a proximal end of the grasper for controlling operation of the grasper;
an external elongated body member connected to the handle member;
an internal elongated body member coupling the handle member and the grasping element;
wherein the external elongated body member comprises a hollow body in which the internal elongated body member is movable from the proximal end to the distal end within the hollow body; and
wherein actuating handles of the handle member control the movement of the internal elongated body member.

5. The grasper of claim 3, wherein the control mechanism is a push-pull lever.

6. The grasper of claim 1, wherein the grasping element comprises:
a top grasping arm;
a plurality of top grasping ridges coupled to the top grasping arm;
a bottom grasping arm;
a plurality of bottom grasping ridges coupled to the bottom grasping arm; and
a control mechanism connecting to the plurality of top grasping edges to extend the plurality of top grasping edges;
wherein the control mechanism is connected to the plurality of bottom grasping edges to extend the plurality of bottom grasping edges.

7. The grasper of claim 6, wherein the plurality of top grasping edges and plurality of bottom grasping edges extend along a longitudinal axis of the grasper.

8. The grasper of claim 6, wherein the plurality of top grasping edges and plurality of bottom grasping edges extend along a direction which is perpendicular to a longitudinal axis of the grasper axis.

9. The grasper of claim 6, further comprising a protruding edge for puncturing a surface.

10. The grasper of claim 1, wherein the grasping element comprises:
a top grasping element;
wherein the top grasping element comprises:
a first top grasping portion;
a second top grasping portion;
a top cavity enabling the first top grasping portion and the second top grasping portion to move when a top wedge is inserted into the top cavity; and
a bottom grasping element; and,
wherein the bottom grasping element comprises:
a first bottom grasping portion;
a second bottom grasping portion; and
a bottom cavity enabling the first bottom grasping portion and the second bottom grasping portion to move when a bottom wedge is inserted into the bottom cavity.

11. The grasper of claim 10, further comprising:
an external elongated body member connected to the handle member;
an internal elongated body member coupling the handle member and the grasping element;
wherein the external elongated body member comprises a hollow body in which the internal elongated body member is movable from the proximal end to the distal end within the hollow body; and
wherein the actuating handles control the movement of the internal elongated body member.

12. A method performed using a grasping element comprising:
configuring the grasping element to a narrow state having a narrow cross-section; inserting the grasping element into an incision in a body;
configuring the grasping element to an expanded state for allowing a grasping operation, the expanded state having an expanded grasping surface area for grasping tissue without damaging said tissue;
upon completion of the grasping operation, configuring the grasping element from the expanded state back to the narrow state; and
removing the grasping element from the incision.

13. The grasper of claim 3, wherein the length of the connecting member determines the grasping surface area of the grasper.

14. The grasper of claim 4, wherein the grasping element is configured to automatically open when it is pushed out the distal end of the external elongated body member, and further wherein the grasping element closes when it is pulled back into the external elongated body member.

15. The grasper of claim 4, wherein the grasping element is controllably openable and closeable.

16. The grasper of claim 14, wherein the grasping element comprises elastically deformable material.

17. The grasper of claim 15, wherein the grasping element comprises selectively actuatable shape memory material.

18. The grasper of claim 4, wherein the grasping element comprises a top grasping element, a bottom grasping element, and a mechanical energy storage device, wherein the mechanical energy storage device is arranged and configured to impart an expanding force on the top and the bottom grasping elements such that the elements are pushed away from each other.

19. The grasper of claim 2, wherein the grasping element comprises:
a first grasping jaw portion;
a second grasping jaw portion;
a connecting member coupling the first grasping jaw portion and the second grasping jaw portion; and
a control mechanism operably coupled to the distal end of the first grasping jaw portion; wherein the first grasping jaw portion rotates around the connecting member when the control mechanism is activated; wherein the rotation causes a distal end of the first grasping jaw portion to pivot around an axis that is perpendicular to a longitudinal axis of the grasper, such that the distal end of the first grasping jaw portion becomes, after completing the rotation, adjacent to a proximal end of the second grasping jaw portion; and
a grasping surface comprising grasping ridges of the first grasping jaw portion adjacent to grasping ridges of the second grasping jaw portion.

20. The grasper of claim 4, wherein the control mechanism is a push-pull lever.
